(19) United States
(12) Patent Application Publication
(10) Pub. No.: US 2009/0318755 A1
(43) Pub. Date: Dec. 24, 2009

(54) THERAPEUTIC PENIS RING

(76) Inventors: Jesse Aaron Adams, Seattle, WA (US); Kenny Chih-yan Tai, Seattle, WA (US); Kevin Tzu-Hsaung Chiu, Bellevue, WA (US)

Correspondence Address: Oliver Law Firm, PS Inc., 1113 Hoyle Ln, Waxhaw, NC, 28173, US

(21) Appl. No.: 12/141,078

(22) Filed: Jun. 18, 2008

(51) Int. Cl. A61F 5/00; A61H 1/00

(52) U.S. Cl. 600/41; 601/46

(57) ABSTRACT

The Therapeutic Vibrator is a small barrel-shaped container housing a motor and at least one battery for the electric power source. The Therapeutic Vibrator is typically activated by an on/off switch or push-button at one end of the barrel, and when activated, the barrel will vibrate delivering massage to a specific body part. The barrel may comprise two halves that fasten together having recesses and cavities for battery (typically button-cell type), motor, switch wiring, etc. The barrel may then be contained within an appropriate vibrator body made from a material that is soft to the touch. The Therapeutic Vibrator may be used in a sex toy or in any other appropriate massaging device, such as small massagers for hands or faces.

### FIELD OF THE INVENTION

This invention pertains generally to therapeutic vibrators, and more specifically to a therapeutic vibrator in the shape of a small barrel being driven by electric means.

### BACKGROUND OF THE FIELD

Vibrators in various forms are often used to stimulate parts of the human body. Specifically, vibrators have been used in sex aids to enhance the sexual experience and satisfaction and/or to minimize sexual dysfunction. In sex applications, it is generally desirable that the vibrator be housed in a housing that is soft and also adapted to be held in an appropriate position. There are some prior art patents that have dealt with these issues—e.g., erectile dysfunction, impotence, and lack of satisfaction.

For instance, U.S. Pat. No. 7,108,668 B2 to Fang discloses a battery-operated vibrator that is housed in a penis ring in a soft housing. However, Fang's device is activated by a sliding switch used to complete a motor circuit. The sliding switch is small and requires friction and a force being applied laterally across the end of the barrel—a manipulation that is difficult to achieve during sexual arousal. Such a sliding switch also effects excessive wear on the internal barrel components (one metal element being chafed against another).

In addition, U.S. Pat. No. 6,790,189 B2 to Kobayashi and U.S. Patent Application No. 2005/0283044 to Chang disclose similar erection support rings which are intended to improve impotence concerns and enhance the sexual experience. However, these devices comprise various additional projections, bumps, and rings and do not disclose such barrel vibrators as in the present invention. Nor do they disclose switching mechanisms. U.S. Patent No. 2006/0247493 to Chen discloses a switching mechanism for activating a vibrating motor. However, Chen's mechanism includes a stick-like design connected to a conductive leaf spring, which could be easily broken or damaged.

### SUMMARY OF THE INVENTION

The present invention solves the above-mentioned problems by providing a vibrating barrel with a push button on/off switch that can activate the vibrator quickly and effectively. The push button switch is easy to manipulate under varying environmental conditions, and can be turned on or off in the same way—enabling the user to effortlessly activate and deactivate the device. Furthermore, the push button switch also allows for less wear to the barrel components because there is minimal friction and the contact (to complete the electrical circuit) is made by direct (z-direction) pressure, not lateral force (x- and y-direction pressure).

One embodiment of the invention comprises a soft ring body which may be made of rubber, soft plastic, other composite or any other suitable material. The ring body may define an open-ended cavity for insertion of a vibrating barrel having a push button on/off switch—displaying a universal on/off symbol—at one end. The push-button switch does not intrude on the intended use, overall shape, and size of the device. The ring body may also comprise a plurality of massaging bumps and knobs protruding therefrom.

The barrel's components are typically aligned along the barrel's longitudinal axis and include a motor and at least one button-cell battery (more in series), in addition to the switch. There will necessarily be connectors to link the battery and the motor. The motor design allows that the invention is extremely durable and conserves battery power and life by its ease of operability that may allow the user to utilize the device a plurality of times before disposal.

### BRIEF DESCRIPTION OF THE DRAWINGS

The objects, features, and advantages of the present invention will be apparent to one skilled in the art from reading the following description in which:

FIG. 1 is a sectional side view of the prior art;

FIG. 2 is a perspective view of the therapeutic penis ring with barrel vibrator installed;

FIG. 3 is an exploded sectional view showing the assembly of the penis ring;

FIG. 4 is a sectional side view of the invention; and

FIG. 5 is a sectional side view of an alternate embodiment of the invention.

### DETAILED DESCRIPTION

The following specification describes a therapeutic penis ring with vibrating barrel and method for using. In the description, specific materials and configurations are set forth in order to provide a more complete understanding of the present invention. But it is understood by those skilled in the art that the present invention can be practiced without those specific details. In some instances, well-known elements are not described precisely so as not to obscure the invention.

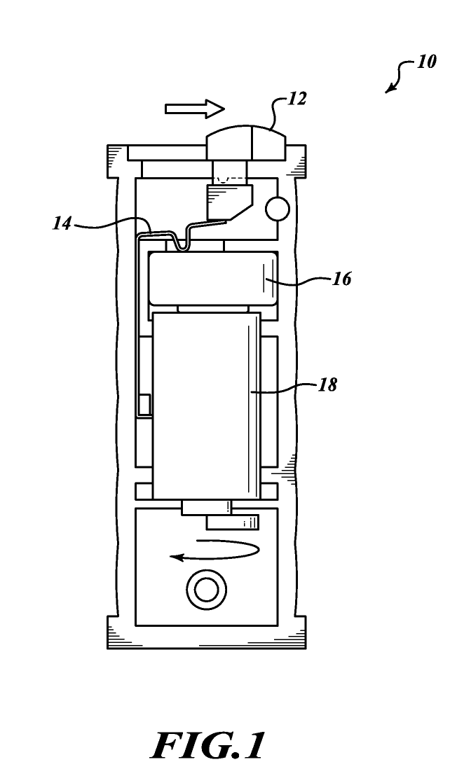

FIG. 1 shows the prior art vibrator 10 with a sliding switch 12. The figure depicts the vibrator 10 in the “on” position with the connector 14 having been activated to complete the circuit between the battery 16 and the motor 18. A user must apply lateral force in the direction of the arrow and at the same time develop friction with the switch 12. Further, sliding the switch on and off repeatedly causes the connector 14 to bend repeatedly and eventually wear so much that it becomes inutile.

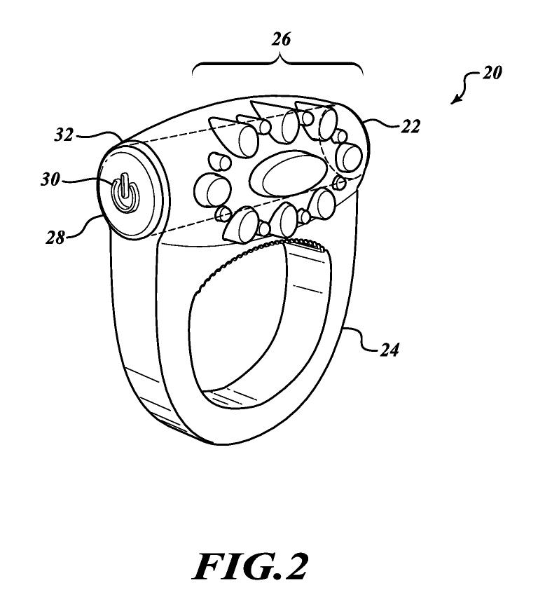

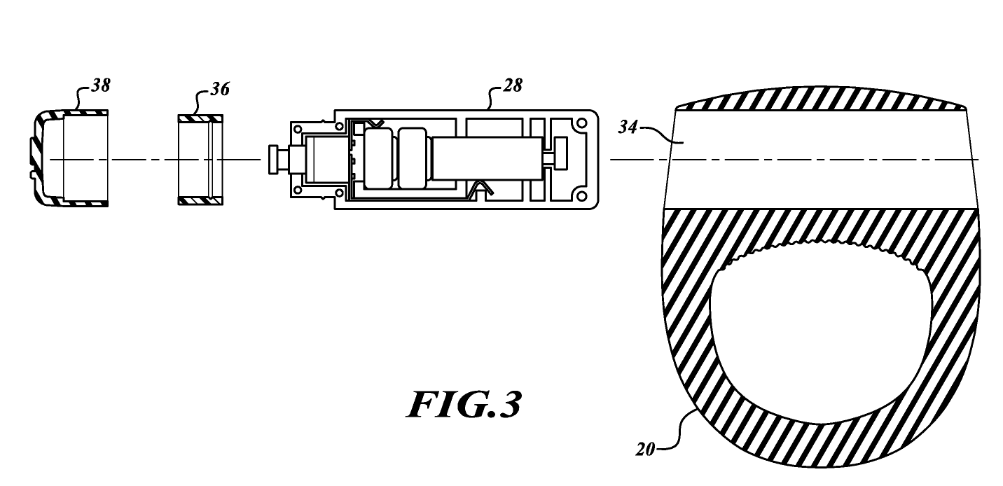

FIG. 2 shows the therapeutic ring 20 of the present invention having a soft vibrator body portion 22 and a soft ring portion 24. The vibrator body 22 defines a generally cylindrical cavity with two open ends designed to snugly hold the generally cylindrical vibrating barrel 28 therein and may also define a plurality of soft exterior bumps (for massage) or other protrusions 26. These protrusions may be arranged on one or both sides of the body portion 22 but are preferably formed in a location corresponding to the vibrator cavity in order to be reliably vibrated. The ring portion 24 has an insert hole through which a penis is to be inserted and elastically constricted while applying stimulation from the vibrating barrel 28 and can obviously be offered in different sizes and shapes. The vibrating barrel 28 comprises a push button on/off switch 30 at a first end 32 and a longitudinal axis 34 (shown in FIG. 3) extending through the first end and the opposing second end.

FIG. 3 shows the ring 20, the barrel 28, and the collar 36 and cap 38 that complete the assembly. The generally cylindrical cavity of the soft body portion of the therapeutic ring is sized to give a slight interference fit with the barrel 28 so that upon insertion, the barrel 28 will be held snugly in place and also out of direct contact with human skin. The generally cylindrical housing of the vibrating barrel 28 may be made of two portions of durable plastic that snap together. These portions may comprise various annular ribs that compartmentalize the cavity of the barrel for holding the various component parts. The generally circular collar 36 encircles the barrel at the first end 32 so as to hold together the two halves, and the soft cap 38 covers the collar and the exposed barrel end so as to provide a soft contact for the user. The soft cap displays a universal on/off symbol for guidance to the user.

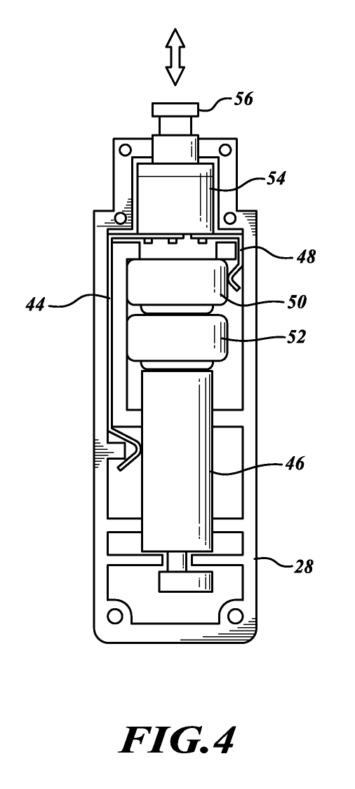

FIG. 4 shows an enlarged sectional view of the barrel 28 comprising a generally cylindrical housing having a motor and at least one button-cell battery therein. The motor connector 44 is in constant contact with the motor 46—thereby eliminating any sliding or frictional wear between the two components. Likewise the battery connector 48 is in constant contact with the battery arrangement, which may include a plurality of (at least one) button-cell batteries in series—here a first battery 50 and a second battery 52. In this preferred configuration shown, the batteries and the motor are configured axially within the barrel 28 and are generally aligned with the longitudinal axis 34.

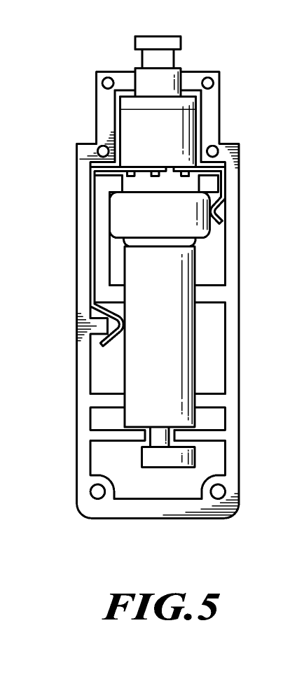

The switch 54 is at least partially contained within the housing and when activated, completes the connection between the motor connector 44 and the battery connector 48 (which are linked to the push button on/off switch), so that the motor begins to vibrate the barrel. The push-button on/off switch comprises a plunger in general axial alignment with the longitudinal axis of the barrel. In operation, the user depresses the plunger 56 by pushing the universal on/off symbol 30 on the cap (shown in FIG. 2) with a direct pushing force, and then can turn off the device by once again pushing the on/off switch 30. Only direct z-pressure is required as shown by the double-headed arrow; no x- or y-forces (lateral forces) are required—making the device useful to a broader range of users and under a broader range of local environmental conditions. The direct pushing force activates the barrel by connecting the motor with the battery through the linked connectors. This design is extremely durable and conserves battery power and life by its ease of operability that may allow the user to utilize the therapeutic device a plurality of times before disposal. FIG. 5 simply shows an alternate embodiment in which the vibrating barrel components are configured with one button-cell battery instead of two. FIG. 5 shows an alternate embodiment of the invention having only one battery.

What is claimed is:

1. A therapeutic penis ring comprising a soft ring adapted to encircle a penis, said ring having a soft vibrator body attached thereto, wherein said vibrator body defines a generally cylindrical cavity for insertion of a vibrating barrel.

2. The therapeutic penis ring of claim 1 wherein said vibrator body defines a plurality of exterior bumps.

3. The therapeutic penis ring of claim 2 wherein said vibrating barrel comprises a push button on/off switch at a first end and defines a longitudinal axis therethrough.

4. The therapeutic penis ring of claim 3 wherein said vibrating barrel further comprises a generally cylindrical housing having a motor and at least one button-cell battery therein.

5. The therapeutic penis ring of claim 4 wherein said at least one battery and said motor are configured axially within said barrel housing and are generally aligned with said longitudinal axis of said barrel.

6. The therapeutic penis ring of claim 5 further comprising a battery connector and a motor connector linked to said push button on/off switch.

7. The therapeutic penis ring of claim 6 wherein said push button on/off switch further comprises a plunger in general axial alignment with said longitudinal axis of said barrel.

8. The therapeutic penis ring of claim 7 wherein said push button on/off switch comprises a universal on/off symbol.

9. An electric vibrating barrel comprising a generally cylindrical housing with a longitudinal axis therethrough, said housing having a first end and an opposing second end.

10. The vibrating barrel of claim 9 wherein said housing comprises two portions snapped together, said portions defining a ribbed cavity.

11. The vibrating barrel of claim 10 further comprising a generally circular collar linked to said first end of said housing and a cap linked to said collar.

12. The vibrating barrel of claim 11 further comprising a motor and at least one battery located within said housing and in general axial alignment with each other and with said longitudinal axis of said barrel.

13. The vibrating barrel of claim 12 further comprising a push button on/off switch being at least partially contained within the housing and activated by direct pushing force being applied to said cap, said switch further being linked to connectors which upon activation connect said motor with said at least one battery.

14. The vibrating barrel of claim 13 wherein said cap displays a universal on/off symbol in general alignment with a plunger of said switch.

15. The vibrating barrel of claim 14 wherein said plunger is activated by direct pushing force.

16. A method of using a vibrating barrel for therapeutic massage of a human body part wherein said vibrating barrel is contained in a soft housing and comprises a motor, at least one battery connected thereto, and a push button on/off switch, said method comprising the steps of selecting a body part, applying said soft housing to said body part, and pushing said push button on/off switch to activate said vibrating barrel.

17. The method of claim 16 wherein said motor, said at least one battery, and said push button on/off switch are generally aligned with each other on a longitudinal axis.

18. The method of claim 16 further comprising the step of pushing said push button on/off switch again to deactivate said vibrating barrel.

19. The method of claim 16 wherein said batteries are button-cell type.

20. The method of claim 16 wherein said push button switch includes a plunger.
